(19) United States
(12) Patent Application Publication
(10) Pub. No.: US 2002/0187904 A1
(43) Pub. Date: Dec. 12, 2002

(54) Detergent cosmetic compositions containing an anionic surfactant derived from amino acids and salts thereof and a silicone and uses thereof

(76) Inventors: Beatrice Perron, Jouy en Josas (FR); Serge Restle, Saint-Prix (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/108,545

(22) Filed: Mar. 29, 2002

(30) Foreign Application Priority Data
Mar. 30, 2001 (FR) 01 04383

(51) Int. Cl. A61K 7/50

(52) U.S. Cl. 510/130; 510/499; 510/477; 510/466

(57) ABSTRACT

Novel cosmetic compositions comprising, in a cosmetically acceptable medium, (a) at least one anionic surfactant chosen from N-acylated compounds of mono- and poly-amidated polycarboxylic amino acids and the salts of said acids and (b) at least one silicone. This combination can impart at least one cosmetic property (such as sleekness, lightness, softness) without keratinous fibre regreasing phenomenon. These compositions are used, for example, for washing and/or conditioning keratinous materials such as the hair or the skin.

The present invention relates to novel cosmetic compositions comprising, in a cosmetically acceptable medium, at least one anionic surfactant chosen from the N-acylated compounds of mono- and polyamidated polycarboxylic amino acids and the salts of said acids and at least one silicone.

The use of detergent compositions (shampoos and shower gels) based essentially on conventional surfactants of the anionic, nonionic and/or amphoteric type, but more particularly of the anionic type, is common for cleansing and/or washing the hair and/or the skin. These compositions are applied to wet hair or skin and the foam generated by massaging or rubbing with the hands allows, after rinsing with water, the removal of the various types of dirt initially present on the hair or the skin.

These base compositions indeed possess good washing power, but the intrinsic cosmetic properties attached to them remain nevertheless fairly weak, in particular because of the fact that the relatively aggressive nature of such a cleansing treatment can cause in the long term damage to the keratinous materials which is marked to a greater or lesser degree, linked in particular to the gradual removal of the lipids or proteins contained in or at the surface thereof.

Accordingly, to improve the cosmetic properties of the above detergent compositions, and more particularly of those that are intended to be applied to sensitive hair (i.e. hair which has become damaged or which has been made fragile especially under the chemical action of atmospheric agents and/or of hair treatments such as permanent waving, dyeing or bleaching), it is now customary to introduce therein additional cosmetic agents called conditioning agents, intended mainly to repair or limit the harmful or undesirable effects induced by the various treatments or attacks to which the hair fibers are subjected more or less repeatedly. These conditioning agents can of course also improve the cosmetic behaviour of natural hair.

The conditioning agents most commonly used to date in shampoos are cationic polymers, silicones and/or silicone-based derivatives, which indeed confer on washed, dry or wet hair greatly improved ease of disentanglement, softness and sleekness compared to what may be obtained with the corresponding cleansing compositions not containing them.

However, in spite of the progress recently made in the field of shampoos based on cationic polymers and/or silicone, the latter are not completely truly satisfactory, such that a high demand currently still exists in relation to being able to have novel products exhibiting better performance at the level of at least one of the cosmetic properties mentioned above.

The N-acylated anionic surfactants of mono- and poly-amidated polycarboxylic amino acids and their salts have already been recommended in detergent cosmetic compositions. They have been described, for example, in patent application WO 97/03171, the disclosure of which is hereby incorporated by reference.

The washing compositions for the hair using these surfactants alone may not lead to completely satisfactory cosmetic properties.

The aim of the invention is therefore to provide detergent cosmetic compositions having at least one improved cosmetic property, such as disentanglement, sleekness and/or softness of the hair.

However, the inventors have now found that the combination of silicones and a particular anionic surfactant makes it possible to achieve at least one of these aims.

These novel compositions can make it possible to better deposit these silicones on a keratinous material such as hair than a composition containing conventional anionic surfactants such as the salts of N-cocoylglutamic acid.

The compositions in accordance with the invention confer on the keratinous materials, in particular the hair, a remarkable treatment effect which manifests itself in particular by at least one of the properties of ease of disentanglement, lightness, sleekness, softness and suppleness with minimal sensation of a charged feel.

The subject of the invention is thus a detergent cosmetic composition, characterized in that it comprises, in a cosmetically acceptable medium, at least one anionic surfactant chosen from the N-acylated compounds of mono- and polyamidated polycarboxylic amino acids and the salts of said acids and at least one silicone.

Another subject of the invention relates to the use of at least one anionic surfactant chosen from the N-acylated compounds of mono- and polyamidated polycarboxylic amino acids and the salts of said acids in, or for the manufacture of a cosmetic composition comprising at least one silicone.

Another subject of the invention relates to a method for treating a keratinous material, such as hair, comprising applying to said materials an effective amount of a cosmetic composition according to the invention. A subject of the invention is also the use of a composition according to the invention in order to increase at least one of disentanglement and sleeking of the hair, in order to impart at least one of volume, lightness, softness, suppleness and manageability on the hair.

According to the present invention, the expression keratinous material is understood to encompass the hair, the eyelashes, the eyebrows, the skin, the nails, the mucous membranes or the scalp, and as a common example the hair.

Various aspects of the invention will now be presented in detail. All the meanings and definitions of the compounds used in the present invention given below are valid for all the aspects of the invention.

### N-acylated Anionic Surfactants of Mono and Polyamidated Polycarboxylic Amino Acids

The N-acylated anionic surfactants of mono- and polyamidated polycarboxylic amino acids and their salts may have the following formula (I):

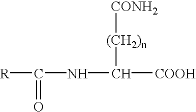

in which:

R is chosen from linear and branched, saturated and unsaturated hydrocarbon radicals comprising from 5 to 29 carbon atoms, for example, R is chosen from mono- and polyunsaturated alkyl and alkenyl radicals comprising from 5 to 29 carbon atoms and as a further example from 7 to 22 carbon atoms, and

n is an integer chosen from 1 and 2.

An additional example would be the N-acylated compounds of mono-and polyamidated polycarboxylic amino acids of formula (I) in which R is chosen from linear, branched, and saturated alkyl radicals comprising from 7 to 29 carbon atoms, such as from 7 to 22 carbon atoms.

The salts of the compounds of formula (I) may be salts of alkali metals (for example sodium and potassium), of alkaline-earth metals (which may be, for example, calcium and magnesium), salts of aqueous ammonia, salts of amines such as those of monoethanolamine, diethanolamine, triethanolamine, 3-amino-1,2-propanediol, and ammonium salts derived from basic amino acids such as lysine, arginine, sarcosine, ornithine, and citrulline.

Among the surfactants of formula (I), there may be mentioned, for example, the salts of N-cocoylglutamine, and in particular the triethanolamine salt of N-cocoylglutamine such as the product marketed under the name FOAM UP DOUCE GM by the company KYOWA HAKKO.

According to the invention, the at least one anionic surfactant chosen from the N-acylated compounds of mono- and polyamidated polycarboxylic amino acids and their salts may, for example, represent from 1% to 30% by weight, such as from 3% to 15% by weight relative to the total weight of the final composition.

Silicones

The silicones which may be used in accordance with the invention may be soluble or insoluble in water or in the final composition. They may be volatile or nonvolatile.

Polyorganosiloxanes are an example of the silicones which may be used in accordance with the invention, the polyorganosiloxanes being insoluble in the composition. Polyorganosiloxanes may be provided in a form chosen from oils, waxes, resins and gums.

The insoluble silicones are dispersed in the compositions in the form of particles generally having a mean size in numerical terms of between 2 nanometres and 100 microns, for example, between 20 nanometres and 20 microns. As will be recognized by one skilled in the art, the mean size of the particles may be measured by a technique suitable for the size measurement being taken, for example, for particles essentially spherical, by scattering techniques such as static light scattering in which a laser granulometer such as MALVERN or an optical particle counter such as Coulter counter by Coultronix may be employed, and dynamic light scattering, in which a laser granulometer such as Brookhaven is employed.

The organopolysiloxanes are defined in greater detail in the book by Walter NOLL “Chemistry and Technology of Silicones” (1968) Academie Press.

The organopolysiloxanes may be volatile or nonvolatile. Volatile silicones may be chosen, for example, from those possessing a boiling point of between 60° C. and 260° C. Additional examples of volatile silicones include those silicones chosen from:

(i) cyclic silicones comprising from 3 to 7 silicon atoms, such as from 4 to 5 silicon atoms. Examples are chosen from the octamethylcyclotetrasiloxane marketed under the name “VOLATILE SILICONE 7207” by UNION CARBIDE and “SILBIONE 70045 V 2” by RHONE POULENC, the decamethylcyclopentasiloxane marketed under the name “VOLATILE SILICONE 7158” by UNION CARBIDE, the decamethylcyclopentasiloxane marketed under the name “SILBIONE 70045 V 5” by RHONE POULENC, and mixtures thereof.

There may also be mentioned cyclocopolymers of dimethylsiloxane/methylalkylsiloxane, such as the “SILICONE VOLATILE FZ 3109” marketed by the company UNION CARBIDE, having the chemical structure:

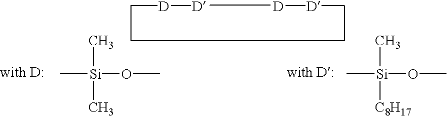

There may also be mentioned mixtures of cyclic silicones with organic compounds derived from silicon, such as the mixture of octamethylcyclotetrasiloxane and tetratrimethylsilylpentaerythritol (50/50) and the mixture of octamethylcyclotetrasiloxane and 1,1′-oxy(2,2,2′,2′,3,3′-hexatrimethylsilyloxy)bisneopentane;

(ii) linear volatile silicones having 2 to 9 silicon atoms and possessing a viscosity of less than or equal to 5×10−6 m^{2 /}s at 25° C. Examples of such linear volatile silicones are chosen from, for example, the decamethyltetrasiloxane marketed under the name “SH 200” by the company TORAY SILICONE. Silicones entering into this class are also described in the article published in Cosmetics and toiletries, Vol. 91, Jan. 76, p. 27-32—TODD & BYERS “Volatile Silicone fluids for cosmetics”.

Non-volatile silicones can also be used in the composition of the invention. Non-volatile silicones are chosen from polyalkylsiloxanes; polyarylsiloxanes; polyalkylarylsiloxanes; silicone gums and resins; polyorganosiloxanes modified by organofunctional groups; the linear polysiloxane(A)-polyoxyalkylene(B) block copolymers of the (A-B)ₙ formula with n>3; graft silicone polymers containing a nonsilicone organic backbone, comprising a principal organic chain formed from organic monomers comprising no silicone, on which there is grafted, inside said chain and optionally at the position of at least one of its ends, at least one polysiloxane macromonomer; graft silicone polymers comprising a polysiloxane backbone grafted with nonsilicone organic monomers, said graft silicone polymers thus comprising a principal polysiloxane chain on which there is grafted, inside said chain and optionally at the position of at least one of the ends of said chain, at least one organic macromonomer comprising no silicone; and mixtures thereof.

Examples of these non-volatile silicones include polyalkylsiloxanes among which there may be mentioned polydimethylsiloxanes with terminal trimethylsilyl groups having a viscosity of 5×10⁻⁶ to 2.5 m²/s at 25° C. such as 1×10⁻⁵ to 1 m^{2/} s. The viscosity of the silicones is, for example, measured at 25° C. according to the ASTM 445 Appendix C standard.

Among these polyalkylsiloxanes, there may be mentioned, without limitation, commercial products chosen from:

SILBIONE oils of the 47 and 70 047 series and MIRASIL oils marketed by the company RHONE POULENC such as the 70 047 V 500 000 oil;

oils of the MIRASIL series marketed by the company RHONE POULENC;

oils of the 200 series from the company DOW CORNING, for example DC200 having a viscosity of 60 000 Cst;

VISCASIL oils from the company GENERAL ELECTRIC and certain oils of the SF series (SF 96, SF 18) also from GENERAL ELECTRIC.

There may also be mentioned the polydimethylsiloxanes with terminal dimethylsilanol groups (Dimethiconol according to the CTFA name), such as the oils of the 48 series marketed by the company RHONE POULENC.

In this class of polyalkylsiloxanes, there may also be mentioned the products marketed under the names “ABIL WAX 9800 and 9801” by the company GOLDSCHMIDT which are poly(C1-C20)alkylsiloxanes.

The polyalkylarylsiloxanes are, for example, chosen from polydimethyl methylphenylsiloxanes and polydimethyl diphenylsiloxanes which are linear and branched and have a viscosity of 1×10⁻⁵ to 5×10⁻² m^{2 /}s at 25°□ C.

Among these polyalkylarylsiloxanes, there may be mentioned, by way of example, products chosen from:

SILBIONE oils of the 70 641 series from the company RHONE POULENC;

oils of the RHODORSIL 70 633 and 763 series from RHONE POULENC;

DOW CORNING 556 COSMETIC GRAD FLUID oil from the company DOW CORNING;

silicones of the PK series from BAYER such as the product PK20;□

silicones of the PN, PH series from BAYER such as the products PN1000 and PH1000; and

certain oils of the SF series from GENERAL ELECTRIC chosen from SF 1023, SF 1154, SF 1250, and SF 1265.

The silicone gums which can be used in accordance with the invention are chosen from polydiorganosiloxanes having high number-average molecular masses ranging from 200 000 to 1 000 000, the polydiorganosiloxanes being able to be used alone or as a mixture in a solvent. This solvent may be chosen from volatile silicones, polydimethylsiloxane (PDMS) oils, polyphenylmethylsiloxane (PPMS) oils, isoparaffins, polyisobutylenes, methylene chloride, pentane, dodecane, tridecane and mixtures thereof.

Additional examples of products are chosen from:

polydimethylsiloxane,

polydimethylsiloxane/methylvinylsiloxane gums,

polydimethylsiloxane/diphenylsiloxane,

polydimethylsiloxane/phenylmethylsiloxane, and

polydimethylsiloxane/diphenylsiloxane/methylvinylsiloxane.

Further examples of products which can be used in accordance with the invention include mixtures chosen from:

mixtures formed from a polydimethylsiloxane hydroxylated at the chain end (called dimethiconol according to the nomenclature of the CTFA dictionary) and from a cyclic polydimethylsiloxane (called cyclomethicone according to the nomenclature of the CTFA dictionary) such as the product Q2 1401 marketed by the company DOW CORNING;

mixtures formed from a polydimethylsiloxane gum with a cyclic silicone such as the product SF 1214 Silicone Fluid from the company GENERAL ELECTRIC, for example, this product is a gum SF 30 corresponding to a dimethicone having a number-average molecular weight of 500 000 that is solubilized in the oil SF 1202 Silicone Fluid corresponding to decamethylcyclopentasiloxane; and

mixtures of two PDMSs of different viscosities, for example a mixture of a PDMS gum and a PDMS oil, such as the product SF 1236 marketed by the company GENERAL ELECTRIC. The product SF 1236 is the mixture of an SE 30 gum defined above having a viscosity of 20 m^{2/}s and an SF 96 oil having a viscosity of 5×10⁻⁶ m²/s. The product SF 1236 comprises approximately 15% of SE 30 gum and 85% of an SF 96 oil.

Examples of organopolysiloxane resins which can be used in accordance with the invention are crosslinked siloxane systems comprising units chosen from R₂SiO_{2/2}, R³SiO_{1/2}, RSiO_{3/2} and SiO_{4/2}, in which R is chosen from hydrocarbon groups comprising from 1 to 16 carbon atoms and phenyl groups. Further examples of organopolysiloxane resins include those crosslinked siloxane systems in which R is chosen from C₁-C₄ lower alkyl radicals such as methyl and phenyl radicals.

Examples of commercial products of organopolysiloxane resins include the product marketed under the name “DOW CORNING 593” by the company DOW CORNING and the products marketed under the names “SILICONE FLUID SS 4230 and SS 4267” by the company GENERAL ELECTRIC which are silicones having the dimethyl/trimethylsiloxane structure.

There may also be mentioned the resins of trimethylsilyloxysilicate which are marketed under the names X22-4914, X21-5034 and X21-5037 by the company SHIN-ETSU.

The organomodified silicones which can be used in accordance with the invention are silicones as defined above comprising in their structure one or more organofunctional groups attached via a hydrocarbon radical.

Among the organomodified silicones, there may be mentioned polyorganosiloxanes comprising at least one of:

polyethyleneoxy and polypropyleneoxy groups optionally comprising C₆-C₂₄ alkyl groups, such as the products called dimethicone-copolyol marketed by the company DOW CORNING under the name DC 1248 and the oils SILWET L 722, L 7500, L 77, and L 711 from the company UNION CARBIDE and the (C₁₂)alkyl-methicone-copolyol marketed by the company DOW CORNING under the name Q2 5200;

substituted and unsubstituted amine-containing groups such as C₁-C₄ aminoalkyl groups, exemplary products are chosen from the products marketed under the name GP 4 Silicone Fluid and GP 7100 by the company GENESEE and the products marketed under the names Q2 8220 and DOW CORNING 929 and 939 by the company DOW CORNING;

quaternary ammonium groups such as the products marketed under the names ABILQUAT 3272 and ABILQUAT 3474 by the company GOLDSCHMIDT;

thiol groups, such as the products marketed under the names “GP 72 A” and “GP 71” from GENESEE;

alkoxylated groups, such as the products marketed under the name “SILICONE COPOLYMER F-755” by the company SWS SILICONES and the products ABIL WAX 2428, 2434 and 2440 marketed by the company GOLDSCHMIDT;

hydroxylated groups, such as the polyorganosiloxanes with a hydroxyalkyl functional group which are described in French patent application FR-A-85 16334, the disclosure of which is hereby incorporated by reference and corresponding to the formula (II):

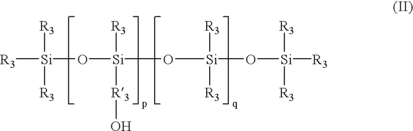

in which the R3 radicals, which are identical or different, are chosen from methyl and phenyl radicals; at least 60 mol % of the R3 radicals comprising methyl; the R′3 radical is chosen from divalent hydrocarbon C2-C18 alkylene members; p is an integer chosen from 1 to 30; and q is an integer chosen from 1 to 150;

acyloxyalkyl groups such as, for example, the polyorganosiloxanes described in U.S. Pat. No.4,957,732,, the disclosure of which is hereby incorporated by reference and corresponding to the formula (X):

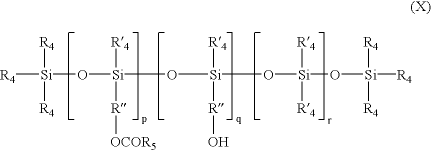

in which:

R₄, which is the same or different, is chosen from methyl, phenyl, —OCOR₅ and hydroxyl groups, provided, however, that only one of the R₄ radicals per silicon atom is OH;

R′₄, which is the same or different, is chosen from methyl and phenyl; wherein at least 60%, as a molar proportion, of all the R₄ and R′₄ radicals comprise methyl;

R₅ is chosen from C₈-C₂₀ alkyl and alkenyl;

R″, which is the same or different, is chosen from linear and branched divalent hydrocarbon C₂-C₁₈ alkylene radicals;

r is an integer chosen from 1 to 120;

p is an integer chosen from 1 to 30;

q is a number chosen from 0 and a number that is less than 0.5 p, wherein the sum of p+q ranges from 1 to 30;

the polyorganosiloxanes of formula (X) may contain groups:

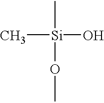

in proportions not exceeding 15% of the sum p+q+r;

anionic groups of the carboxylic type, such as, for example, in the products described in patent EP 186 507 from the company CHISSO CORPORATION, and anionic groups of the alkylcarboxylic type such as those present in the product X-22-3701 E from the company SHIN-ETSU; 2-hydroxy-alkylsulphonate;

hydroxyalkylthiosulphate such as the products marketed by the company GOLDSCHMIDT under the names “ABIL S201” and “ABIL S255”;

hydroxyacylamino groups, such as the polyorganosiloxanes described in application EP 342 834. There may be mentioned, for example, the product Q2-8413 from the company DOW CORNING.

The block copolymers having a linear polysiloxane-polyoxyalkylene block of the (A-B)n type used in the context of the present invention have the following formula:

([Y(R₂SiO)ₐR′₂SiYO][(CₙH₂ₙO)_{b}])_{c} (V)

in which:

R and R′, which are identical or different, are chosen from monovalent hydrocarbon radicals containing no aliphatic unsaturation;

n is an integer chosen from 2 to 4;

a is chosen from integers greater than or equal to 5, for example integers from 5 to 200 such as integers from 5 to 100;

b is chosen from integers greater than or equal to 4, for example integers from 4 to 200 such as integers from 5 to 100;

c is chosen from integers greater than or equal to 4, for example integers from 4 to 1 000 such as integers from 5 to 300; and

Y represents a divalent organic group which is linked to the adjacent silicon atom by a carbon-silicon bond and to a polyoxyalkylene block by an oxygen atom.

the mean molecular weight of each siloxane block is from about 400 to about 10,000, the mean molecular weight of each polyoxyalkylene block being from about 300 to about 10,000,

the siloxane blocks comprise from about 10% to about 95% by weight of the block copolymer,

the weight-average molecular weight of the block copolymer is at least 3,000, for example, from 5,000 to 1,000,000 and as a further example from 10,000 to 200,000,

An example of a block copolymer of formula (V) is one in which R and R′ are chosen from the group comprising alkyl radicals such as methyl, ethyl, propyl, butyl, pentyl, hexyl, octyl, decyl and dodecyl radicals; aryl radicals such as phenyl and naphthyl radicals; aralkyl radicals such as benzyl and phenylethyl radicals; tolyl radicals; xylyl radicals and cyclohexyl radicals;

Y is chosen from —R″—, —R″—CO—, —R−—NHCO—, 13 R″—NH—CO—NH—R′″—NHCO—, and —R″—OCONH—R′″—NHCO—, where R″ is a divalent alkylene group chosen from ethylene, propylene and butylene and R′″ is chosen from divalent alkylene groups and divalent arylene groups such as —C₆H₄—, —C₆H₄—C₆H₄—, —C₆H₄—CH₂—C₆H₄— and C₆H₄—C(CH₃)₂—C₆H₄—.

As a further example Y is chosen from divalent alkylene radicals such as the radical —CH₂—CH₂—CH₂— and the radical C₄H₈.

The preparation of the block copolymers used in the context of the present invention is described in European application EP 0,492,657 A1, the disclosure of which is hereby incorporated by reference.

According to the invention, it is also possible to use silicone-containing polyurethanes such as those described in patent applications EP 0,751,162, EP 0,619,111 and EP 1,025,833, the disclosures of which are hereby incorporated by reference.

According to the invention, it is also possible to use silicones comprising a polysiloxane portion and a portion comprising a nonsilicone organic chain, wherein one of the two portions constitutes the principal chain of the polymer and the other portion is grafted onto said principal chain. These polymers are for example described in patent applications EP-A-412 704, EP-A-412 707, EP-A-640 105 and WO 95/00578, EP-A-582 152 and WO 93/23009 and patents U.S. Pat. No. 4,693,935, U.S. Pat. No. 4,728,571 and U.S. Pat. No. 4,972,037, the disclosures of which are hereby incorporated by reference.. These polymers may be chosen, for example, from anionic polymers and nonionic polymers.

Such polymers are, for example, the copolymers which can be obtained by free-radical polymerization starting with a mixture of monomers, said mixture of monomers comprising:

a) 50 to 90% by weight of tert-butyl acrylate;

b) 0 to 40% by weight of acrylic acid;

c) 5 to 40% by weight of a silicone-containing macromer of formula:

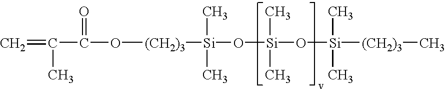

in which v is an integer chosen from 5 to 700; wherein the percentages by weight are calculated relative to the total weight of the monomers.

Other examples of graft silicone-containing polymers are chosen from polydimethylsiloxanes (PDMS) on which are grafted, via a linking member of the thiopropylene type, mixed polymer units of the poly(meth)acrylic acid type and of the polyalkyl (meth)acrylate type, and polydimethylsiloxanes (PDMS) on which are grafted, via a linking member of the thiopropylene type, polymer units of the polyisobutyl (meth)acrylate type.

According to the invention, all the silicones may also be used in forms chosen from emulsions, nanoemulsions and microemulsions.

As additional examples are provided polyorganosiloxanes chosen from:

nonvolatile silicones chosen from the family of polyalkylsiloxanes with terminal trimethylsilyl groups, said polyalkylsiloxanes with terminal trimethylsilyl groups chosen from oils having a viscosity of between 0.2 and 2.5 m2/s at 25° C. such as the oils of the DC200 series from the company DOW CORNING, particularly those oils of the DC200 series having a viscosity of 60 000 Cst, the oils of the series SILBIONE 70047 and 47 and the oil 70 047 V 500 000 marketed by the company RHONE POULENC, polyalkylsiloxanes with dimethylsilanol terminal groups such as dimethiconol, and polyalkylarylsiloxanes such as the SILBIONE 70641 V 200 oil marketed by the company RHONE POULENC;

the organopolysiloxane resin marketed under the name DOW CORNING 593;

polysiloxanes with amino groups such as amodimethicones and trimethylsilylamodimethicone; and

polysiloxanes containing quaternary ammonium groups.

According to the invention the silicone(s) may represent, for example, from 0.001 % to 15% by weight, for further example, from 0.01% to 10% by weight and as an even further example from 0.1 to 5% by weight, relative to the total weight of the final composition.

The compositions of the invention may also contain, in addition, at least one other surfactant which is generally, for example, present in a quantity from 0.1% to 40% by weight approximately, for example from 3% to 30% and as a further example from 5% tod 20%, relative to the total weight of the composition.

This surfactant may be chosen from anionic, amphoteric and nonionic surfactants, and mixtures thereof.

Additional surfactants for carrying out the present invention can be chosen from the following:

(i) Anionic surfactant(s):

Their nature may not be of truly critical importance within the context of the present invention.

Thus, by way of example of anionic surfactants that can be employed, by themselves or as mixtures, in the context of the present invention, there may be mentioned (nonlimiting list) salts (for example alkali metal salts such as sodium, ammonium salts, amine salts, amino alcohol salts and magnesium salts) of compounds:chosen from alkyl sulphates, alkyl ether sulphates, alkylamido ether sulphates, alkylaryl-polyether sulphates, monoglyceride sulphates, alkyl sulphonates, alkyl phosphates, alkylamidesulphonates, alkyl aryl sulphonates, (α-olfinsulphonates, paraffin-sulphonates, alkyl sulphosuccinates, alkyl ether sulphosuccinates, alkylamidesulphosuccinates, alkyl sulphosuccinamates, alkyl sulphoacetates, alkyl ether phosphates, acyl sarcosinates, acyl isethionates and N-acyltaurates, the alkyl or acyl radical of all these different compounds containing from 8 to 24 carbon atoms, and the aryl radical chosen from phenyl and benzyl groups. Among the anionic surfactants which may be used in the invention there may also be mentioned the salts of acids chosen from oleic, ricinoleic, palmitic and stearic fatty acids, acids of copra oil and of hydrogenated copra oil, and acyl lactylates in which the acyl radical contains 8 to 20 carbon atoms. Weakly anionic surfactants may also be employed such as those chosen from alkyl-D-galactosideuronic acids and the salts of said acids, polyoxyalkylenated carboxylic (C6-C24)alkyl ether acids, polyoxyalkylenated carboxylic (C6-C24)alkylaryl ether acids, polyoxyalkylenated carboxylic (C6-C24)alkyl amidoether acids and the salts of said acids, such as those containing from 2 to 50 ethylene oxide groups, and mixtures thereof.

Additional examples of anionic surfactants include those chosen from the salts of alkyl sulphates and of alkyl ether sulphates and mixtures thereof.

(ii) Nonionic surfactant(s):

The nonionic surfactants themselves are also compounds which are well known per se (in this respect see the “Handbook of Surfactants” by M. R. Porter, published by Blackie & Son (Glasgow and London), 1991, pp. 116-178) and, in the context of the present invention, their nature may not be of critical importance. They can thus be chosen from (nonlimiting list) alcohols, alpha-diols, alkylphenols, and polyethoxylated, polypropoxylated and polyglycerolated fatty acids which have a fatty chain containing, for example, 8 to 18 carbon atoms, it being possible for the number of ethylene oxide or propylene oxide groups to range from 2 to 50 and it being possible for the number of glycerol groups to range from 2 to 30. The copolymers of ethylene oxide and propylene oxide and the condensates of ethylene oxide and propylene oxide with fatty alcohols may also be mentioned by way of example. Further examples are chosen from polyethoxylated fatty amides containing for example from 2 to 30 mol of ethylene oxide, polyglycerolated fatty amides containing for example 1 to 5 glycerol groups, such as 1.5 to 4, polyethoxylated fatty amines containing for example 2 to 30 mol of ethylene oxide, oxyethylenated fatty acid esters of sorbitan containing, for example, from 2 to 30 mol of ethylene oxide, fatty acid esters of sucrose, fatty acid esters of polyethylene glycol, alkylpolyglycosides, N-alkylglucamine derivatives, amine oxides such as the oxides of (C10-C14) alkylamines, and N-acylaminopropylmorpholine oxides. Alkylpolyglycosides are still another example of nonionic surfactants for the present invention.

(iii) Amphoteric surfactant(s):

The additional amphoteric surfactants, the nature of which may not be of critical importance in the context of the present invention, may be chosen from (nonlimiting list) derivatives of aliphatic secondary and tertiary amines in which the aliphatic radical is chosen from linear and branched chains containing 8 to 22 carbon atoms and containing at least one water-solubilizing anionic group (for example carboxylate, sulphonate, sulphate, phosphate and phosphonate); (C8-C20)alkylbetaines, sulphobetaines, (C8-C20)alkylamido(C1-C6)alkylbetaines and (C8-C20)alkylamido(C1 -C6)alkylsulphobetaines.

Among the amine derivatives there may be mentioned the products sold under the name Miranol, as described in U.S. Pat. No. 2,528,378 and U.S. Pat. No. 2,781,354 and of structures chosen from:

R2 —CONHCH2 CH2 —N(R3)(R4)(CH2COO—) (2)

in which R2 is chosen from alkyl radicals derived from R2—COOH acids present in hydrolysed copra oil, such as a heptyl, nonyl and undecyl radical; R3 is a beta-hydroxyethyl group; and R4 is chosen from a carboxymethyl group and

R₅—CONHCH₂CH₂—N(B)(C) (3)

in which:

(B) represents —CH2CH2OX′;

(C) represents —(CH2)z−Y′, wherein z is an integer chosen from 1 and 2;

X′ is chosen from —CH2CH2—COOH and a hydrogen atom;

Y′ is chosen from —COOH and —CH2—CHOH—SO3H; and

R5 is chosen from alkyl radicals of a carboxylic acid present in copra oil and in hydrolysed linseed oil such as radicals chosen from C7, C9, C11, C13, and C17 alkyl radicals, iso C17 alkyl radicals and saturated C17 radicals.

These compounds are classified in the CTFA dictionary, 7th edition, 1997, under the names Disodium Cocoamphodiacetate, Disodium Lauroamphodiacetate, Disodium Capryloamphodiacetate, Disodium Caproamphodiacetate, Disodium Cocoamphodipropionate, Disodium Lauroamphodipropionate, Disodium Caproampho-dipropionate, Disodium Capryloamphodipropionate, Lauro-amphodipropionic acid, and Cocoamphodipropionic acid.

By way of example, there may be mentioned the commercial product of disodium cocoamphodiacetate marketed under the trade name MIRANOL® C2M concentrate by the company RHODIA CHIMIE.

Thus, representative compositions of the invention may include mixtures of surfactants chosen from mixtures of anionic surfactants, mixtures of anionic surfactants and amphoteric surfactants and mixtures of anionic surfactants and nonionic surfactants.

Examples of such mixtures of surfactants include additional anionic surfactants chosen from sodium, triethanolamine and ammonium (C₁₂-C₁₄)alkyl sulphates, oxyethylenated sodium, triethanolamine and ammonium (C₁₂-C₁₄)alkyl ether sulphates containing 2.2 mol of ethylene oxide, sodium cocoyl isethionate, sodium (C₁₄-C₁₆)alphaolefin sulphonate and mixtures of sodium cocoyl isethionate and sodium (C₁₄-C₁₆)alphaolefin sulphonate with an amphoteric surfactant chosen from:

amphoteric surfactants such as the amine derivatives called disodium cocoamphodipropionate and sodium cocoamphopropionate marketed by the company RHODIA CHIMIE under the trade name “MIRANOL® C2M CONC” in aqueous solution at 38% of active material and under the name MIRANOL® C32, and amphoteric surfactants such as alkylbetaines, for example cocobetaine marketed under the name “DEHYTON® AB 30” in aqueous solution at 32% AS by the company HENKEL and the (C8-C20)alkylamido(C1-C6)alkylbetaines, such as TEGOBETAINE® F 50 marketed by the company GOLDSCHMIDT.

Anionic surfactant(s) different from the N-acylated compounds of mono- and polyamidated polycarboxylic amino acids and their salts are generally, for example, present in an amount of 1 to 30% by weight, for example from 3 to 15% by weight, relative to the total weight of the composition.

The amphoteric and nonionic surfactant(s) are generally, for example, present in an amount of about 0.5 to about 15% by weight, for example from 1 to 5% by weight, relative to the total weight of the composition.

The quantity and the quality of the surfactants are those sufficient to confer at least one of the qualities of satisfactory foaming and satisfactory detergent power on the final composition.

In the composition according to the present invention, all the detergent surfactants generally, for example, represent from 4 to 50% by weight, for example, from 6 to 35% by weight and as a further example from 8 to 25% by weight, relative to the total weight of the composition.

The composition of the invention may also contain at least one additive chosen from thickeners, perfumes, pearlescent agents, preservatives, sunscreens, cationic surfactants, anionic, nonionic, and amphoteric polymers, cationic polymers, proteins, protein hydrolysates, ceramides, pseudoceramides, linear and branched chain C16-C40 acids such as 18-methyleicosanoic acid, hydroxy acids, vitamins, provitamins such as panthenol, vegetable oils, mineral oils and synthetic oils, antidandruff agents and any other additive conventionally used in the cosmetic field which does not affect the stability and the properties of the compositions according to the invention.

These additives may be present in the composition according to the invention in proportions which may range from 0.001 to 50% by weight relative to the total weight of the composition. The precise quantity of each additive is easily determined by a person skilled in the art according to its nature and its function.

According to one embodiment of the invention, the compositions according to the invention comprise, in addition, one or more cationic polymers.

The cationic polymers which can be used in accordance with the present invention may be chosen from all those already known per se to improve the cosmetic properties of hair treated with detergent compositions, namely those described in patent application EP-A-0,337,354 and in French patent applications FR-A-2,270,846, 2,383,660, 2,598,611, 2,470,596 and 2,519,863, the disclosures of which are hereby incorporated by reference.

More generally still, for the purposes of the present invention, the expression “cationic polymer” denotes any polymer containing cationic groups and/or groups which can be ionized into cationic groups.

Among the cationic polymers capable of being used in the context of the present invention, use may be made of cationic polymers chosen from quaternary cellulose ether derivatives such as the products marketed under the name “JR 400” by the company UNION CARBIDE CORPORATION, cyclopolymers, for example the homopolymers of diallyidimethylammonium salt and the copolymers of diallyidimethylammonium salt and acrylamide, such as the chlorides, which are marketed under the names “MERQUAT 100”, “MERQUAT 550” and “MERQUAT S” by the company MERCK, cationic polysaccharides, and guar gums modified with 2,3-epoxypropyltrimethylammonium chloride which are marketed for example under the name “JAGUAR C13S” by the company MEYHALL.

It is also possible to use the polymers which comprise repeating units corresponding to the formula:

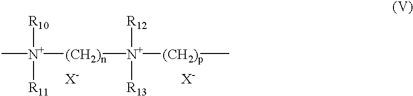

in which R10, R11, R12 and R13, which are identical or different, are chosen from alkyl and hydroxyalkyl radicals having from about 1 to 4 carbon atoms, n and p are chosen from integers ranging from about 2 to 20 and X— is chosen from anions derived from inorganic acids and anions derived from organic acids.

According to the invention, the cationic polymer(s) may, for example, represent from 0.001% to 10% by weight, for example from 0.005% to 5% by weight, and as a further example from 0.01% to 3% by weight, of the total weight of the final composition.

The cosmetically acceptable medium may be chosen from water and mixtures of water and a cosmetically acceptable solvent. The cosmetically acceptable solvent may be chosen from lower C1-C4 alcohols, such as ethanol, isopropanol, tert-butanol and n-butanol and alkylene glycols such as propylene glycol and glycol ethers.

For example, the composition may, for example, comprise from 50 to 95% by weight of water relative to the total weight of the composition.

The detergent compositions according to the invention have a final pH which is generally, for example, from 3 to 10, for example, a pH from 4 to 8. The adjustment of the pH to the desired value may be carried out conventionally by addition of a base chosen from organic bases and inorganic bases to the composition, for example a base chosen from aqueous ammonia and primary, secondary and tertiary (poly)amines such as monoethanolamine, diethanolamine, triethanolamine, isopropanolamine and 1,3-propanediamine. The adjustment of the pH may also be carried out conventionally by addition of an acid, for example a carboxylic acid such as citric acid.

The compositions in accordance with the invention may contain, in addition to the combination defined above, viscosity-regulating agents chosen from electrolytes and thickening agents which may be chosen from sodium chloride, scleroglucans, xanthan gums, fatty acid alkanolamides, alkyl ether carboxylic acid alkanolamides which are optionally oxyethylenated with up to 5 mol of ethylene oxide, such as the product marketed under the name “AMINOL A15” by the company CHEM Y, crosslinked polyacrylic acids and crosslinked acrylic acid/C10-C30 alkyl acrylate copolymers. These viscosity-regulating agents are used in the compositions according to the invention in proportions which may be up to 10% by weight relative to the total weight of the composition.

The compositions in accordance with the invention may also contain up to 5% of pearlescent and opacifying agents well known in the state of the art, such as, those chosen from sodium and magnesium palmitates, sodium and magnesium stearates, sodium and magnesium hydroxystearates, fatty chain-containing acylated derivatives such as ethylene glycol and polyethylene glycol monostearates and distearates, and fatty chain-containing ethers such as distearyl ether and 1 -(hexadecyloxy)-2-octadecanol.

The compositions according to the invention may also contain foam synergists such as those chosen from C10-C18 1,2-alkanediols and fatty alkanolamides derived from mono- or diethanolamine.

The compositions in accordance with the invention may be used for washing and treating a keratinous material chosen from hair, skin, eyelashes, eyebrows, nails, lips, scalp and as a common example, the hair.

In particular, the detergent compositions according to the invention are chosen from shampoos, shower gels and foam baths.

The compositions of the invention may also be provided in forms chosen from rinse-off and leave-in after-shampoos, compositions for permanent waving, straightening, dyeing and bleaching, and rinse-off compositions for permanent waving, straightening, dyeing and bleaching which may be applied at times chosen from before and after dyeing, bleaching, permanent waving or straightening and between the two stages of permanent waving and straightening.

The compositions of the invention may also be provided in the form of make-up removing products.

The compositions according to the invention may be provided in forms chosen from gels, milks, creams, emulsions, thickened lotions and thickened foams and may be used for a keratinous material chosen from skin, scalp, nails, eyelashes, lips and more particularly the hair.

These detergent compositions may be foaming and the foaming power of the compositions according to the invention, characterized by a foam height, is generally greater than 75 mm, preferably greater than 100 mm measured according to the modified ROSS-MILES method (NF T 73-404/ISO696).

The modifications of the method are the following:

The measurement is carried out at the temperature of 22° C. with osmosed water. The concentration of the solution is 2 g/l. The drop height is 1 m. The quantity of composition which drops is 200 ml. These 200 ml of composition fall into a measuring cylinder having a diameter of 50 mm and containing 50 ml of the composition tested. The measuring is made 5 minutes after stopping the flow of the composition.

The subject of the invention is also a method for treating a keratinous material such as skin and hair, characterized in that it comprises applying to the keratinous material an effective amount of a cosmetic composition as defined above, and then in optionally rinsing, such as with water.

Thus, the method according to the invention allows for at least one of treatment of, care of, washing of and removal of make-up from a keratinous material such as skin hair and any other keratinous material.

In the text which follows or in the preceding text, the percentages expressed are by weight.

The invention will now be illustrated more fully with the aid of the following examples which cannot be considered as limiting the embodiments described. In the examples, AS means active substance.

### EXAMPLE 1:

Three shampoo compositions were prepared, one in accordance with the invention (composition A) and the other 2 comparatives (compositions B and C):

| | A | | |
|---|---|---|---|
| | (inven- | | |
| | tion) | B | C |
| - Triethanolamine salt of N-cocoyl | 16.7 g | — | — |
| glutamine at 30% AS (Foam up | (5 gAS) | | |
| doucé GM from KYOWA HAKKO) | | | |
| - Cocoylbetaine as an aqueous | 8.3 g | 8.3 g | 8.3 g |
| solution at 30% AS (DEHYTON AB | (2.5 gAS) | (2.5 gAS) | (2.5 gAS) |
| 30 from COGNIS) | | | |
| - Sodium (C₁₂-C₁₄)alkyl ether | 38 g | 38 g | 57 g |
| sulphate oxyethylenated with 2.2 | (9.9 gAS) | (9.9 gAS) | (14.8 |
| mol of ethylene oxide as an | | | gAS) |
| aqueous solution at 26% of AS | | | |
| -N-cocoyl glutamate as an aqueous | — | 16.7 g | — |
| solution at 30% AS (Acyl glutamate | | 5 gAS | |
| CT12 from AJINOMOTO) | | | |
| - Copra acid | 2.5 g | 2.5 g | 2.5 g |
| monoisopropanolamide | | | |
| - 1-(Hexadecyloxy)-2- | 2.5 g | 2.5 g | 2.5 g |
| octadecanol/cetyl alcohol mixture | | | |
| - Polydimethylsiloxane of viscosity | 1.5 g | 1.5 g | 1.5 g |
| 500,000 cSt (MIRASIL DM 500,000 | | | |
| from RHODIA CHIMIE) | | | |
| - Sodium chloride | 1 | 2 | |
| - Preservatives | qs | qs | qs |
| - pH agents qs pH | 6.5 | 6.5 | 6.5 |
| - Demineralized water qs | 100 g | 100 g | 100 g |

Shampooing was performed by applying about 1 g of composition A to locks of 2.5 g of previously wet natural hair. The shampoo was caused to lather, an exposure time of 10 minutes allowed to elapse and then the locks were abundantly rinsed off with water. The locks were dried for 10 minutes at 60° C. A second application of the composition was carried out.

The same procedure as above was used with the comparative compositions B and C.

Experts compared the locks in pairs.

### Composition A/Composition C

A panel of experts evaluated the appearance of the wet hair.

90% of the experts preferred the lock treated with composition A and indicated that the hair treated with composition A according to the invention is significantly sleeker and/or softer than that treated with composition C.

### Composition A/Composition B

A panel of experts evaluated the appearance of the wet hair.

90% of the experts preferred the lock treated with composition A and indicated that the hair treated with composition A according to the invention is significantly sleeker than that treated with composition B.

We claim:

1. A detergent cosmetic composition comprising, in a cosmetically acceptable medium, (a) at least one anionic surfactant chosen from N-acylated compounds of mono- and polyamidated polycarboxylic amino acids and the salts of said acids and (b) at least one silicone.

2. The composition of claim 1 wherein said at least one anionic surfactant is chosen from N-acylated compounds of mono- and polyamidated polycarboxylic amino acids of the following formula (I): 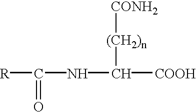
wherein:
R is chosen from linear and branched, saturated and unsaturated hydrocarbon radicals comprising from 5 to 29 carbon atoms, and
n is an integer chosen from s and 2.

3. The composition of claim 2 wherein R is chosen from mono- and polyunsaturated alkyl and alkenyl radicals comprising from 5 to 29 carbon atoms.

4. The composition of claim 3 wherein the alkyl and alkenyl radicals comprise from 7 to 22 carbon atoms.

5. The composition of claim 1, wherein said at least one anionic surfactant is an N-cocoylglutamine salt.

6. The composition of claim 1 wherein the at least one silicone is chosen from oils, waxes, resins and gums.

7. The composition of claim 1, wherein the at least one silicone comprises polyorganosiloxanes chosen from polyalkylsiloxanes, polyarylsiloxanes, polyalkylarylsiloxanes, silicone gums, silicone resins, and polyorganosiloxanes modified by organofunctional groups.

8. The composition of claim 7, wherein the polyalkylsiloxanes are chosen from:
polydimethylsiloxanes with terminal trimethysilyl groups;
polydimethylsiloxanes with terminal dimethylsilanol groups; and
polyalkyl (C₁-C₂₀)siloxanes;
the polyalkylarylsiloxanes are chosen from polydimethylmethylphenylsiloxanes;
the silicone gums are chosen from polydiorganosiloxanes having number-average molecular masses from 200 000 to 1 000 000;
the silicone resins are chosen from silicone resins comprising units: R₃ Si O1/2, R₂ Si O2/2, R Si O3/2, and Si O4/2
wherein R is chosen from hydrocarbon groups comprising from 1 to 16 carbon atoms and phenyl groups; and
the organomodified silicones are chosen from silicones comprising one or more organofunctional groups attached via a hydrocarbon radical.

9. The composition of claim 7 wherein the silicone gums are chosen from:
polydimethylsiloxanes;
polydimethylsiloxane/methylvinylsiloxanes;
polydimethylsiloxaneld iphenylsiloxanes;
polydimethylsiloxane/phenylmethylsifoxanes;
polydimethylsiloxane/diphenylsiloxane/methylvinylsiloxanes; and mixtures chosen from:
mixtures formed from a polydimethylsiloxane hydroxylated at the chain end and a cyclic polydimethylsiloxane;
mixtures formed from a polydimethylsiloxane gum and from a cyclic silicone; and
mixtures of polydimethylsiloxanes of different viscosities.

10. The composition of claim 7, wherein the organomodified silicones are chosen from polyorganosiloxanes comprising at least one group chosen from:
polyethyleneoxy and polypropyleneoxy groups;
substituted and unsubstituted amino groups;
thiol groups;
alkoxylated groups;
hydroxyalkyl groups;
acyloxyalkyl groups;
alkyl carboxylic groups;
2-hydroxyalkyl sulphonate groups;
2-hydroxyalkyl thiosulphate groups;
hydroxyacylamino groups; and
quaternary ammonium groups.

11. The composition of claim 7 wherein the polyorganosiloxanes are chosen from polyalkylsiloxanes with terminal trimethylsilyl groups, polyalkylsiloxanes with terminal dimethylsilanol groups, polyalkylarylsiloxanes, mixtures of more than one polydimethylsiloxane chosen from gums and oils of different viscosities, mixtures formed from a polydimethylsiloxane hydroxylated at the chain end and a cyclic polydimethylsiloxane, mixtures formed from a polydimethylsiloxane gum with a cyclic silicone, organopolysiloxane resins and polysiloxanes with amino groups.

12. The composition of claim 1 wherein the at least one silicone is chosen from polyorganosiloxanes which are insoluble in the composition.

13. The composition of claim 1 wherein the at least one anionic surfactant is present at a concentration from 1 to 30% by weight relative to the total weight of the composition.

14. The composition of claim 1 wherein the at least one anionic surfactant is present at a concentration from 3 to 15% by weight relative to the total weight of the composition.

15. The composition of claim 1 wherein the at least one silicone is present at a concentration from 0.001% to 15% by weight relative to the total weight of the composition.

16. The composition of claim 1 wherein the at least one silicone is present at a concentration from 0.01% to 10% by weight relative to the total weight of the composition.

17. The composition of claim 1 wherein the at least one silicone is present at a concentration from 0.1% to 5% by weight relative to the total weight of the composition.

18.The composition of claim 1 wherein the composition further comprises at least one additional surfactant chosen from anionic, cationic, nonionic and amphoteric surfactants.

19. The composition of claim 18 wherein the at least one additional surfactant is present at a concentration from 0.1% to 40% by weight relative to the total weight of the composition.

20. The composition of claim 18 wherein the at least one additional surfactant is present at a concentration from 3% to 30% by weight relative to the total weight of the composition.

21. The composition of claim 18 wherein the at least one additional surfactant is present at a concentration from 5% to 20% by weight relative to the total weight of the composition.

22.The composition of claim 1 wherein the composition further comprises at least one additive chosen from thickeners, perfumes, pearlescent agents, preservatives, sunscreens, anionic polymers, nonionic polymers, amphoteric polymers, cationic polymers, proteins, protein hydrolysates, ceramides, pseudoceramides, linear and branched chain C₁₆-C₄₀ acids, hydroxy acids, vitamins, provitamins, vegetable oils, mineral oils, synthetic oils and antidandruff agents.

23. The composition of claim 22 wherein the linear and branched chain C₁₆-C₄₀ acids are chosen from 18-methyleicosanoic acid.

24. The composition of claim 22 wherein the provitamins are chosen from panthenol.

25. The composition of claim 22 wherein the cationic polymers are chosen from;
homopolymers of diallyldimethylammonium salt and copolymers of dimethylammonium salt and acrylamide;
quaternary cellulose ether derivatives;
cationic polysaccharides;
polymers comprising repeating units corresponding to the formula: 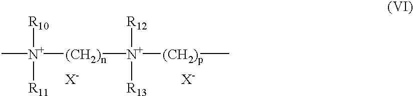
in which R₁₀, R₁₁, R₁₂ and R₁₃, which are identical or different, are chosen from alkyl and hydroxyalkyl radicals having from about 1 to 4 carbon atoms, n and p are chosen from integers ranging from 2 to 20 and X⁻ is chosen from anions derived from inorganic acids and anions derived from organic acid.

26. A shampoo or a washing composition for the skin,a rinse-off or leave-in after-shampoo, a composition for permanent waving, straightening, dyeing or bleaching, or a rinse-off composition for permanent waving, straightening, dyeing or bleaching comprising, in a cosmetically acceptable medium, (a) at least one anionic surfactant chosen from N-acylated compounds of mono- and polyamidated polycarboxylic amino acids and the salts of said acids and (b) at least one silicone.

27. A method for washing a keratinous material comprising applying to said keratinous material an effective amount of a composition comprising in a cosmetically acceptable medium, (a) at least one anionic surfactant chosen from N-acylated compounds of mono and polyamidated polycarboxylic amino acids and the salts of said acids and (b) at least one silicone.

28. A method for increasing the disentanglement or sleeking of hair comprising applying to the hair an effective amount of a composition comprising, in a cosmetically acceptable medium, (a) at least one anionic surfactant chosen from N-acylated compounds of mono and polyamidated polycarboxylic amino acids and the salts of said acids and (b) at least one silicone, said applying occurring for a time sufficient to impart at least one of volume, lightness, softness, suppleness or manageability to the hair.

29. A method for treating a keratinous material comprising applying to said materials an effective amount of a cosmetic composition and then optionally rinsing off, wherein said cosmetic composition comprises, in a cosmetically acceptable medium, (a) at least one anionic surfactant chosen from N-acylated compounds of mono and polyamidated polycarboxylic amino acids and the salts of said acids and (b) at least one silicone.

30. The method of claim 29 wherein said keratinous material is hair.
